(19) United States
(12) Patent Application Publication
(10) Pub. No.: US 2025/0281319 A1
(43) Pub. Date: Sep. 11, 2025

(54) Motorized Penis Extender

(71) Applicant: Baseem Najjar, Lewes, DE (US)

(72) Inventor: Baseem Najjar, Lewes, DE (US)

(21) Appl. No.: 19/087,447

(22) Filed: Mar. 22, 2025

Related U.S. Application Data
(63) Continuation-in-part of application No. 18/595,798, filed on Mar. 5, 2024, now abandoned.

(51) Int. Cl. A61F 5/41

(52) U.S. Cl. CPC A61F 5/41; A61F 2005/412

(57) ABSTRACT

According to an aspect of the present invention, there is provided device for enlargement of the penis of a user, comprising: a motor; a tension sensor; a hook for holding a vacuum cup that attaches to the head of the penis; a bottom semi-circular member; a motor wheel configured to be driven by the motor; a rod with a track connected to the motor wheel; and a button for controlling the motor wheel, wherein: the motor wheel is configured to run along the track on the rod.

### INCORPORATION BY REFERENCE

U.S. Pat. No. 11,918,505B1 is hereby incorporated by reference in its entirety.

### BACKGROUND

Methods and devices for enhancing the penis of a male individual are well-known in the patent literature.

For example, US20150202109 discloses a penis enlargement device comprising a substantially flat member with a hole and neck adapted to fit over a human penis to be positioned flat against a user's lower abdomen; a ring member also adapted to fit around the base of the penis; a waist strap attached to said flat member adapted to hold the flat member in place; a substantially cylindrical cover attached to said neck adapted to connect to either stretch cords or to a weight container; whereby, weight or force is applied to the base of the penis causing the penis to enlarge over time.

U.S. Pat. No. 7,802,577 discloses a harness for stretching the penis, includes a belt for encircling the waist of the user, with a fastening mechanism secured to a rear part of the belt. A first tractive means applies tractive forces at the base of the penis of a user of the harness, and is secured to the belt via the fastening mechanism. A second tractive means applies tractive forces at the edge of the head of the penis of a user of the harness, and is also secured to the belt via the fastening mechanism. The first tractive means and the second tractive means apply a stretching force to the penis of the user of the harness.

U.S. Pat. No. 8,075,473 discloses an extension device for permanent penis enlargement and straightening via long-term elongation, the extension device comprising: a fastening means for application to a penis, the fastening means having an accommodation body, the accommodation body: being dimensionally stable, accommodating a glans penis of the penis over the entire surface of the glans penis when the glans penis is inserted into the extension device, having an inner contour essentially corresponding to a shape of the glans penis, having an opening for allowing insertion of the glans penis, and having an inner surface; a pulling apparatus connected to the fastening means; an elastic tube seal connected to the opening of the accommodation body; and a lubricant located on the inner surface of the accommodation body; wherein the pulling apparatus is formed by: a support, strap, belt or other clothing accessory or piece of clothing, the support, strap, belt or other clothing accessory or piece of clothing being connected to the accommodation body, being held on a body of the user, and being used as a stop, a stationary object, a frame supported on a body of the user, the frame being adjustable in length, or a weight attached to the accommodation body.

Nonetheless, prior art methods and devices suffer from significant drawbacks, including physical danger and inefficiency in bringing about the desired penile enlargement.

### SUMMARY OF INVENTION

Therefore, the present invention provides an improved device for enlarging the penis of a user.

According to an aspect of the present invention, there is provided According to an aspect of the present invention, there is provided device for enlargement of the penis of a user, comprising: a motor; a tension sensor; a hook for holding a vacuum cup that attaches to the head of the penis; a bottom semi-circular member; a motor wheel configured to be driven by the motor; a rod with a track connected to the motor wheel; and a button for controlling the motor wheel, wherein: the motor wheel is configured to run along the track on the rod.

### BRIEF DESCRIPTION OF THE DRAWINGS

FIG. 1 illustrates a penis enlargement device according to an embodiment of the present invention.

### DETAILED DESCRIPTION

The present invention is an automatic motorized penis extender.

The idea is to have a penis extender that has a small motor in it and sensors that will allow to either set tension manually by using the buttons or keep the device adjusting tension automatically by setting a specific tension in the app for it to aim at.

### Workflow:

The user attached his penis using a vacuum cup into the extender.

Then he can press the up down buttons, the button turns on the motor and it starts twisting the metal rod moving the cup holder and the cup up/down
this will result in increasing or decreasing tension on the penis.

The cup holder has a tension sensor and can display that tension in real time on the app.

The user can set desired tension and the app will turn on the motor to increase/decrease tension by increasing/decreasing the length of the device.

The device also has a magnetic sensor which can determine what is the length of the cup holder from the base and it can calculate the length of the penis in the extender

Those details are transmitted to the app in real time and also saved locally on the device.

The device can show how the penis expanded over time in the extender and the app can show the growth of the penis over days and months because it's collecting that data at all times.

The app can suggest target tension for the user in order to ensure optimal expansion and growth.

The app also collects data from all users to determine which parameters are allowing for maximum growth so that it can use those for auto learning and future suggestions.

The user can hold the device horizontally using a strap that can connect to the top ears on the extender.

Embodiments illustrative of the present invention will be described with reference to the attached drawings.

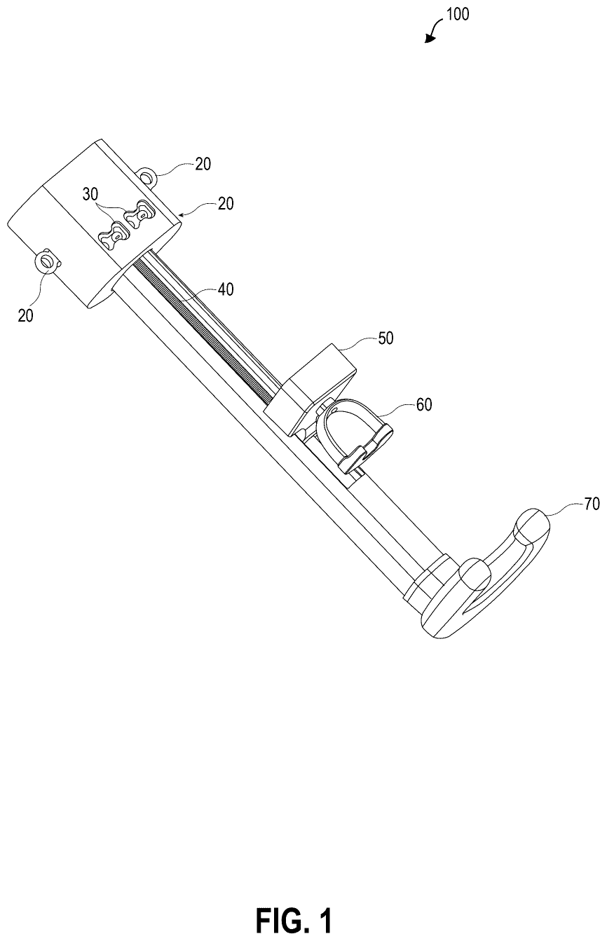

FIG. 1 illustrates a penis enlargement device according to an embodiment of the present invention.

The following components are illustrated:
Device 100 refers to the penis enlargement device according to an embodiment of the present invention as a whole.
Up/down button 30 controls motor 10.
Strap Holder 20 allows the user to hold the device.
Motor 10 has a wheel for providing electromotive penis extending force which runs along a track on rod 40.
Rod 40 is the structural component of the device.
Tension sensor 50 measures the tension on the penis.
Cup holder 60 allows for the device to be used with a penis extender cup. A penis extender cup can be used with the device of embodiments to maximize the penis enlargement experience by a user of the device.
Silicon base 70 is a bottom semi-circular member configured to comfortably rest on a user's groin.

The embodiments described above are given merely for example and for the purpose of facilitating the understanding of the present invention and are not intended to limit the interpretation of the present invention. The respective elements and their arrangements, materials, conditions, shapes, sizes, or the like of the embodiment are not limited to the illustrated examples but may be appropriately changed. Further, the constituents described in the embodiment may be partially replaced or combined together.

What is claimed is:

1. A device for enlargement of the penis of a user, comprising:
a motor;
a tension sensor;
a hook for holding a vacuum cup that attaches to the head of the penis;
a bottom semi-circular member;
a motor wheel configured to be driven by the motor;
a rod with a track connected to the motor wheel; and
a button for controlling the motor wheel, wherein:
the motor wheel is configured to run along the track on the rod.
